(19) United States
(12) Patent Application Publication
(10) Pub. No.: US 2025/0332372 A1
(43) Pub. Date: Oct. 30, 2025

(54) NO SUPPLY INSTALLATION COMPRISING AN NO DELIVERY APPARATUS FED BY GAS CYLINDERS

(71) Applicant: INOSYSTEMS, Antony (FR)

(72) Inventors: Yann BLANDIN, Antony (FR); Frederic MARCHAL, Antony (FR); Nicolas TIRILLY, Antony (FR); Nathan PROUVEZ, Antony (FR)

(21) Appl. No.: 19/175,067

(22) Filed: Apr. 10, 2025

(30) Foreign Application Priority Data
Apr. 24, 2024 (FR) 2404233

(51) Int. Cl. A61M 16/12; A61M 16/00; A61M 16/20

(52) U.S. Cl. CPC A61M 16/12; A61M 16/009; A61M 16/201; A61M 2016/0027; A61M 2202/0275; A61M 2205/123

(57) ABSTRACT

The invention relates to an installation (100) for supplying an NO-containing gas mixture to a patient, comprising NO containers (10.1, 10.1) containing an NO-containing gas at a given pressure, and an NO delivery apparatus (1) for supplying the NO-containing gas, comprising control means (210), a gas circuit (200) with circuit sections (201, 202) comprising gas inlets (201.1, 202.1) for fluidically connecting the containers (10.1, 10.2) thereto, and also pressure-measuring means (251, 252) for measuring the pressure there, and valve means (222.1, 222.2) controlled by the control means (210) in order to control the flow of gas within the circuit sections (201, 202). The control means (210) control the valve means (222.1, 222.2) in order to authorize circulation of gas in one of the sections (201, 202) and simultaneously to prohibit all circulation of gas in the other of the sections (201, 202) as long as the measured pressure (P) is greater than or equal to a given threshold pressure, then to switch from one section to the other when the pressure falls below the given threshold pressure. Preferably, a step is provided for purging the section receiving the gas before switching to said section.

### TECHNICAL FIELD

The invention relates to an installation for supplying an NO-based gas mixture to a patient, comprising an NO delivery apparatus for supplying an NO-containing gas mixture, typically an NO/N₂ mixture, from one or more NO sources, such as pressurized gas cylinders, and a medical ventilator supplying an oxygen-based gas (i.e. >20 vol % approximately), such as air or an O₂/N₂ mixture, so as to obtain a combined gas containing NO and oxygen.

### BACKGROUND

Inhaled nitric oxide (NO or iNO) is a gaseous medicament commonly used to treat patients suffering from acute pulmonary hypertension, in particular pulmonary vasoconstrictions in adults or children, including newborns (PPHN), as described for example in EP-A-560928 or EP-A-1516639.

To implement therapy by inhaled NO, use is made of a gas supply installation, also known as an NO administration installation, comprising an NO delivery apparatus and a medical ventilator, that is to say a respiratory assistance apparatus, supplying a patient circuit.

The NO delivery apparatus makes it possible to inject a gas mixture based on NO, typically an NO/nitrogen mixture, into the patient circuit, which is also supplied with a gaseous flow containing oxygen (at least approximately 20 vol %), such as air or an oxygen/nitrogen mixture (O₂/N₂), supplied by the medical ventilator. The patient circuit generally comprises one or more flexible conduits which are fluidically connected to a respiratory interface, such as a tracheal intubation tube or the like, which is used to supply to the patient to be treated a therapeutic gas mixture containing a given quantity or dose of NO, that is to say a dosage, typically of between 5 and 40 ppmv of NO.

A gas supply installation of this kind is described by EP3821929, for example. This type of installation is used in a hospital environment in order to administer the treatment by NO and thereby to care for patients who need to inhale NO in order to treat their pulmonary arterial hypertension. Other installations of this type are described by EP4209243, EP4241817, EP4241812 and EP4295882.

The NO gas, typically an NO/nitrogen gas mixture, supplied to the NO delivery apparatus generally comes from one or more pressurized gas containers, i.e. one or more gas cylinders containing the compressed NO/nitrogen gas mixture, also called an “NO cylinder”.

In order to avoid interruption of treatment of the patient when an NO cylinder is empty, i.e. during the time required to replace it with a full NO cylinder, it is recommended that the NO delivery apparatus be fluidically connected to two NO cylinders. Thus, when an NO cylinder is (almost) empty, a caregiver can actuate a switching valve or similar in order to cut off the supply from the empty NO cylinder and authorize the supply from the full NO cylinder, so as to ensure continuity of the NO fluid supply to the NO delivery apparatus and to allow the empty NO cylinder to be replaced with a full one.

A problem that arises in practice is that caregivers may either forget to check the pressure displayed by the pressure gauge, usually fitted to an NO cylinder, and therefore fail to notice that a cylinder is (almost) empty, or they may not be available to perform these operations because they are too busy treating a patient. In both cases, there is a risk of interruption of the NO supply, which is not acceptable because it could potentially endanger the patient.

WO2015/172160 and US2023270960 propose a management of gaseous NO supplies from NO cylinders of an NO supply installation, based on the calculation of a gas autonomy, called run-time-to-empty, that is to say a time of use before the total emptying of each cylinder. The calculation is based on monitoring the residual pressure level in the NO cylinders. This approach is not ideal because it requires continuously measuring the pressure in the gas cylinders themselves, which complicates the overall architecture of the installation.

In view of this, an object of the invention is to be able to avoid or minimize this risk of the NO supply being interrupted due to non-replacement of an (almost) empty NO cylinder of an installation for supplying an NO-based gas mixture to a patient, so as to improve the safety of treatment of a patient treated by administration of NO gas.

### SUMMARY

A solution according to the invention concerns an installation for supplying an NO-containing gas mixture to a patient, comprising:
A) a first and a second NO container, such as gas cylinders, containing an NO-containing gas at a given pressure,
B) an NO delivery, i.e supply, apparatus or device for supplying the NO-containing gas, typically an NO/N₂ mixture, comprising:
   control means, preferably microprocessor-based control means, such as an electronic controller or similar,
   a gas circuit comprising:
      a first circuit section comprising a first gas inlet configured to fluidically connect thereto the first NO container containing the NO-containing gas, and
      a second circuit section comprising a second gas inlet configured to fluidically connect thereto the second NO container containing the NO-containing gas,
   a first pressure-measuring means, arranged on the first circuit section, configured to measure the pressure within the first circuit section,
   a second pressure-measuring means, arranged on the second circuit section, configured to measure the pressure within the second circuit section,
   a first valve means, arranged on the first circuit section, controlled by the control means in order to control the flow of gas within the first circuit section, that is to say to authorize/allow or, conversely, prohibit/stop all circulation of gas within the first circuit section, and
   a second valve means, arranged on the second circuit section, controlled by the control means in order to control the flow of gas within the second circuit section, that is to say to authorize/allow or, conversely, prohibit/stop all circulation of gas within the second circuit section,
C) first and second expansion means arranged upstream of the NO delivery apparatus and configured to reduce the pressure of the NO-containing gas from the first and second NO containers to a given expansion pressure (PD) of less than 10 bar, and
D) a first gas feed line and a second gas feed line, respectively, for conveying the NO-containing gas, which has passed through the expansion means (i.e. the first and second expansion means), to the first gas inlet of the first circuit section and to the second gas inlet of the second circuit section, respectively.

Moreover, the control means of the NO delivery apparatus of an installation according to the invention are configured to control the first valve means and/or the second valve means to:
i) authorize circulation of gas within one of the first and second circuit sections and simultaneously prohibit circulation of gas within the other of said first and second circuit sections as long as the pressure (P) measured (e.g. the instantaneous pressure) by the first or second pressure-measuring means is greater than or equal to a given threshold pressure (PS), i.e. P≥PS, and
ii) when the pressure (P) measured by the first or second pressure-measuring means falls below the given threshold pressure (PS), i.e.

$$P < PS :$$

a) interrupt all circulation of gas within said first or second circuit section in which gas circulates, i.e. in one of the two sections, and
b) authorize circulation of gas within the other of said first or second circuit sections within which circulation of gas was prohibited, that is to say in the other of the two sections,
   where said given threshold pressure (PS) is lower than the expansion pressure (PD), i.e. PS<PD.

In other words, in the apparatus according to the invention, there is an automatic changeover from one section to the other, as soon as the pressure measured within the section in question is less than or equal to the given threshold pressure (PS), which corresponds to an empty or almost empty gas cylinder, in particular when the measured pressure falls below a threshold pressure (PS) of the order of 3 to 4 bar.

The gas supply is therefore carried out alternately, depending on the pressure prevailing in the first and second circuit sections.

Thus, the control means of the NO delivery apparatus are configured to control the first valve means and/or the second valve means to:
authorize/allow circulation of gas within the first circuit section and simultaneously prohibit all circulation of gas within the second circuit section as long as the pressure (P) measured by the first pressure-measuring means is greater than or equal to the given threshold pressure (PS),
then, when the pressure (P) measured by the first pressure-measuring means falls below the given threshold pressure (PS), to interrupt/stop all circulation of gas within said first circuit section in which gas was previously circulating, and then authorize circulation of gas within the second circuit section within which the circulation of gas was previously prohibited/prevented.

Thereafter, either reciprocally or alternatively, the control means of the NO delivery apparatus are configured to control the first valve means and/or the second valve means to:
authorize/allow circulation of gas within the second circuit section and simultaneously prohibit all circulation of gas within the first circuit section as long as the pressure (P) measured by the second pressure-measuring means is greater than or equal to the given threshold pressure (PS),
then, when the pressure (P) measured by the second pressure-measuring means falls below the given threshold pressure (PS), to interrupt/stop all circulation of gas within said second circuit section in which gas was circulating, and then authorize circulation of gas within the first circuit section within which the circulation of gas was prohibited/prevented.

In other words, the NO delivery, i.e. supply, apparatus or device serving to supply an NO-containing gas in an installation according to the invention comprises:
control means, and
a gas circuit comprising at least two circuit sections arranged preferably in parallel, each comprising:
   a gas inlet configured to fluidically connect thereto an NO container containing the NO-containing gas, and
   a pressure-measuring means configured to measure the gas pressure therein, and
   a valve means controlled by the control means in order to control the flow of gas therein, that is to say to authorize/allow or, conversely, prohibit/stop all circulation of gas within the section concerned,
      and in which the control means are configured to control one of the valve means to:
authorize circulation of gas within one of the sections and simultaneously prohibit all circulation of gas within the other of said sections as long as the pressure (P) measured by the pressure-measuring means associated with the section in question is greater than or equal to a given threshold pressure (PS), i.e.

$$P \geq PS ,$$

and when the measured pressure (P) falls below the given threshold pressure (PS), within the section in question:
a) interrupt all circulation of gas within the section in question in which gas was circulating, i.e. where a flow was hitherto passing, and
b) authorize circulation of gas within the other section within which the circulation of gas was prohibited, that is to say in the other of the sections, i.e. the section in which the gas flow was hitherto interrupted.

Advantageously, before authorizing circulation of gas within the other section, said section is purged.

Depending on the embodiment in question, the installation according to the invention for NO-containing gas mixture can comprise one or more of the following features:
the first circuit section and the second circuit section of the apparatus for delivering NO, typically an NO/N₂ mixture, are connected to each other at a connection site of the gas circuit located downstream of the first and second valve means.
the first and second valve means of the NO delivery apparatus comprise solenoid valves.
the first gas inlet of the NO delivery apparatus is configured to be fluidically connected to the first NO container via a first gas feed line.
the second gas inlet of the NO delivery apparatus is configured to be fluidically connected to the second NO container via a second gas feed line.
the first gas feed line and the second gas feed line comprise flexible hoses or the like.
first pressure-reducing means are arranged upstream of the first gas inlet of the first circuit section.
second pressure-reducing means are arranged upstream of the second gas inlet of the second circuit section.
the first pressure-reducing means and/or the second pressure-reducing means comprise at least one gas expansion device, that is to say a pressure-reducing device configured to reduce, i.e. decrease, the pressure of the gas passing through it, here the pressure of the NO-based gas mixture.
the first pressure-reducing means and/or the second pressure-reducing means are configured to reduce (i.e. control) the pressure of the NO-containing gas to an expansion pressure (PD) of less than 10 bar, typically of the order of 4 to 8 bar, preferably between 4 and 7 bar, more preferably between 4 and 6 bar, advantageously between 5 and 6 bar.
the first pressure-reducing means are arranged downstream of the first NO container containing the NO-based gas, typically an NO/N₂ mixture.
the second pressure-reducing means are arranged downstream of the second NO container containing the NO-based gas, typically an NO/N₂ mixture.
when the NO containers are full, the NO/N₂ mixture contained therein is at a pressure (i.e. a starting pressure) of at least 150 bar, referred to as “high pressure”. This high pressure corresponds to the pressure of the NO-based gas before expansion, e.g. the NO/N₂ mixture, that is to say before pressure reduction by passage through the first and/or second pressure-reducing means.
the first gas container is equipped with a first gas distribution valve serving to control the supply of NO-based gas from the first gas container, i.e. the gas distribution.
the second gas container is equipped with a second gas distribution valve serving to control the supply of NO-based gas from the second gas container, i.e. the gas distribution.
according to one embodiment, the first and/or second pressure-reducing means are arranged downstream, i.e. at the outlet, of the first and second distribution valves arranged on the first and second gas containers, respectively.
according to another embodiment, the first and/or the second pressure-reducing means are integrated in the distribution valves arranged on the first and second gas containers, respectively, that is to say that said gas distribution valves are valves with integrated pressure reducers (RDI).
the first gas inlet of the apparatus is configured to be fluidically connected to the first valve of the first NO container via the first gas feed line.
the second gas inlet of the apparatus is configured to be fluidically connected to the second valve of the second NO container via the second gas feed line.
the first pressure-reducing means are arranged between a gas outlet of the first valve of the first NO container and a gas inlet of the first gas feed line.
the second pressure-reducing means are arranged between a gas outlet of the second valve of the second NO container and a gas inlet of the second gas feed line.
the NO-based gas undergoes a decrease in pressure to the expansion pressure (PD) by passing through the first pressure-reducing means or the second pressure-reducing means.
the NO-based gas flows through the first and second gas feed lines in the direction from the NO containers to the delivery apparatus, i.e. in the direction of the first and second gas inlets.
the NO-based gas fed by the first and/or second gas feed line is at the expansion pressure (PD).
the apparatus additionally comprises a first exhaust line to the atmosphere comprising a first exhaust valve, fluidically connected to the first circuit section.
the apparatus additionally comprises a second exhaust line to the atmosphere comprising a second exhaust valve, fluidically connected to the second circuit section.
the first and second exhaust valves of the apparatus are controlled by the control means.
the first exhaust line to the atmosphere and the second exhaust line to the atmosphere communicate with the atmosphere via one or more exhaust ports of the apparatus.
the exhaust of the gas to the ambient atmosphere takes place via a single exhaust port or alternatively via several exhaust ports of the apparatus, for example two exhaust ports.
the control means of the apparatus are additionally configured to control the second exhaust valve in order to purge the second circuit section, before authorizing gas from the second NO container to circulate within said second circuit section.
alternatively, the control means of the apparatus are additionally configured to control the first exhaust valve in order to purge the first circuit section, before authorizing gas from the first NO container to circulate within said first circuit section.
the control means of the apparatus are additionally configured to purge the first or second circuit section and simultaneously at least a part of the first or second gas feed line connected to said purged first or second circuit section, that is to say one or more flexible hoses.
the control means are additionally configured to perform a purge between steps a) and b) above.
the control means are additionally configured to perform a purge for a given purge duration.
the purge duration is between 10 seconds and 120 seconds, typically at least 30 seconds.
the purge duration is stored, for example by storage means of the apparatus.
the purging of the first and/or second circuit section comprises sending or discharging to the atmosphere at least some of the pressurized gas (i.e. at the residual pressure) present in the first or second circuit section of the apparatus and preferably in at least part of the first or second gas feed line connected to said first or second circuit section subjected to purging of the apparatus.
the purging of the first and/or second circuit section comprises sending or discharging to the atmosphere at least some of the pressurized gas and at least some of the unwanted species that may be present therein, in particular harmful or toxic species, typically NO₂ species.
the purging comprises a gas sweep with the NO-containing gas mixture, typically the NO/N₂ mixture, coming from the first or second NO container.
the purging of the first and/or second circuit section comprises a gas sweep (at least) of said first and/or second circuit section with the NO/N₂ gas mixture, i.e. the NO-based gas coming from one or other of the gas containers.
the purging comprises a gas sweep of said first or second circuit section, respectively, and of said first or second gas feed line, respectively, i.e. a gas sweep of the first section and of the first gas feed line associated with it, or of the second section and of the second gas feed line associated with it, with the NO/N₂ gas mixture, i.e. the NO-based gas coming from one or other of the gas containers, i.e. the first or second gas container, respectively.
sending or discharging to the atmosphere at least some of the gaseous atmosphere (i.e. gas containing any NO₂ impurities) present in the first or second circuit section of the apparatus and in the first or second gas feed line, namely purge gas containing the NO/N₂ mixture used for the gas sweep and possibly any impurities, such as the NO₂ species.
the given threshold pressure (PS) is stored by storage means of the apparatus.
the given threshold pressure (PS) is between 2 and 5 bar, preferably between 2 and 4 bar, more preferably between 3 and 4 bar.
the first NO container and the second NO container contain an NO/N₂ mixture containing between 100 and 2000 ppmv of NO, and nitrogen for the remainder.
the first NO container and the second NO container contain an NO/N₂ mixture containing between 100 and 1500 ppmv, typically between 200 and 1000 ppmv.
the apparatus is supplied with a gaseous mixture formed by nitrogen and NO.
the apparatus comprises dose adjustment means which are configured to allow a user to fix or select the set NO content corresponding to the desired final proportion of NO in the combined gaseous mixture, i.e. a dosage.
the dose adjustment means form part of an HMI (human-machine interface) or GUI (graphical user interface).
the dose adjustment means of the apparatus comprise one or more touch keys which can be actuated by the user and are displayed on a digital touch screen of the HMI, preferably of the type displaying in colour.
the set NO content is between 1 and 80 ppmv, typically between 5 and 40 ppmv.
the storage means of the apparatus comprise a computer memory, such as a flash memory, a RAM or the like.
the control means comprise a (micro)controller or the like.
the control means comprise one or more (micro)processors arranged on one or more electronic boards.
the control means comprise one or more (micro)processors which implement one or more algorithms, in particular one or more algorithms for controlling valves, for processing flow or pressure measurements, etc.
the storage means are arranged on the electronic board.
it is supplied electrically by one or more electric current sources, typically the mains supply (110/220 V) and/or one or more rechargeable batteries.
the NO delivery apparatus is supplied with NO-containing gas from two gas containers alternately supplying one or other of the gas circuit sections of the apparatus, typically two cylinders of pressurized gas.
the installation comprises a medical ventilator configured to supply an O₂-containing respiratory gas flow, such as air or an O₂/N₂ mixture.
the installation comprises a respiratory circuit comprising an injection device which is configured to mix the NO-containing gas from the NO delivery apparatus with the flow of O₂-containing respiratory gas supplied by the medical ventilator, and to obtain a combined gas mixture containing NO and oxygen.
the NO delivery apparatus and the medical ventilator are fluidically connected to the injection device arranged on the respiratory circuit.
the medical ventilator is configured to supply a flow of respiratory gas containing at least 20 vol % approximately of O₂, typically an NO/N₂ mixture or air.
the NO delivery apparatus and the medical ventilator are fluidically connected to the respiratory circuit, in particular via the injection device.
a flow sensor is arranged in the respiratory circuit between the medical ventilator and the injection device.
the injection device comprises a first gas inlet, which is supplied with a respiratory gas flow containing O₂, i.e. coming from the medical ventilator.
the injection device additionally comprises a second gas inlet, which is supplied with gas containing NO at the said set flow, i.e. coming from the NO delivery apparatus.
the injection device additionally comprises a gas outlet supplying the combined gaseous mixture containing NO and oxygen, obtained by mixing, within the injection device, the gas containing NO (e.g. NO/N₂ mixture) with the flow of respiratory gas containing O₂ (e.g. air or O₂/N₂ mixture).
the medical ventilator delivers air or an oxygen/nitrogen mixture, i.e. as a respiratory gas containing at least 20 vol % approximately of oxygen, preferably at least 21 vol % approximately of oxygen.
the medical ventilator comprises a motorized blower (i.e. turbine, compressor or similar) delivering the respiratory gas, typically air or an oxygen/nitrogen mixture or, according to another embodiment, an internal gas circuit comprising one or more proportional valves for conveying the gas and controlling its supply, in particular its flow rate. Such a ventilator is generally supplied with respiratory gas via one or more wall outlets supplied with gas from a network of channels in a hospital building, typically with air or an oxygen/nitrogen mixture.
the medical ventilator comprises control means or a control device, such as one or more electronic control boards. Preferably, the control means of the medical ventilator control or command the motorized blower or, depending on the circumstances, the proportional valves of the medical ventilator.
the medical ventilator is of the HFO type or comprises an HFO function, that is to say it is able to produce high-frequency oscillations.
each NO container contains an NO/N₂ gas mixture containing between 100 and 2000 ppmv of NO, the remainder being nitrogen (N₂), preferably between 100 and 1000 ppmv of NO.
each NO container contains an NO/N₂ gas mixture conditioned at a pressure (measured before start of withdrawal) of between 10 and 250 bar, preferably of more than 50 bar, typically of more than 100 bar.
each NO container is or comprises one or more gas cylinders with a capacity of between 0.5 and 50 l (water equivalent).
each NO container comprises a cylindrical body made of steel or of aluminium alloy.
each NO container is equipped with what is called a “simple” gas distribution valve (without integrated expander) or with an “integrated expander” (RDI) valve incorporating expansion means (i.e. pressure reduction means), preferably an RDI.
each gas distribution valve (i.e. simple or RDI) is protected by a protective cap, for example made of metal or polymer.
each NO container is equipped with a “simple” valve (i.e. without an integrated expander), and a gas expansion device is arranged downstream of each valve.
the pressure-reducing means, i.e. the gas expansion means, are arranged downstream of the “simple” valve of each NO container containing the NO-based gas, typically an NO/N₂ mixture, so as to decrease or reduce the pressure of the NO-based gas, typically an NO/N₂ mixture, leaving the valve in question, i.e. each valve, and thus obtain the gas at the desired expansion pressure (PD) of less than 10 bar.
the pressure-reducing means comprise or are (at least) a gas expansion device, more simply called an “expander”.
each expander is fixed to a (simple) valve by being fluidically connected to the outlet of said valve, for example by screwing or the like.
alternatively, the pressure-reducing means are arranged in each valve fitted to each NO container, i.e. are integrated into each RDI-type valve, so as to decrease or reduce the pressure of the NO-based gas, typically an NO/N₂ mixture, before it leaves the valve in question, i.e. each valve.
in all cases, the pressure-reducing means are configured to reduce the pressure of the NO-based gas, typically an NO/N₂ mixture, to an expansion pressure of less than or equal to 10 bar, preferably less than 8 bar, typically between 4 and 7 bar, for example between 5 and 6 bar.
the gas feed lines, typically the first and second gas feed lines, are connected downstream of each RDI or of each expansion valve.
the gas feed lines, typically the first and second gas feed lines, are configured to convey the NO-based gas after its pressure has been reduced to the expansion pressure (PD) of less than or equal to 10 bar, i.e. the expanded gas.
the gas feed lines, typically the first and second gas feed lines, comprise flexible hoses, typically made of polymer.
the gas outlet of each valve (simple or RDI) is arranged on an outlet connection, such as a nozzle or similar.
the respiratory circuit of the installation comprises an inspiratory branch and an expiratory branch, typically flexible conduits forming the inspiratory branch and the expiratory branch, for example hoses made of polymer.
the inspiratory branch and the expiratory branch, e.g. flexible conduits, are connected to a joining piece, such as a Y-piece.
the inspiratory branch and/or the expiratory branch are fluidically connected to a patient respiratory interface, preferably via the joining piece.
the patient respiratory interface comprises a tracheal intubation tube or a breathing mask or the like.
the inspiratory branch and the expiratory branch are moreover fluidically connected to outlet and inlet orifices, respectively, of the medical ventilator.
the respiratory circuit, in particular the inspiratory branch, can comprise a gas humidifier.
the gas humidifier is arranged downstream of the injection device, for example an NO injection module, so as to be able to humidify the gas before it is administered to the patient by inhalation.
the medical ventilator is supplied electrically by one or more electric current sources, typically the mains supply (110/220 V) and/or one or more rechargeable batteries.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstrictions or similar, said method comprising administration by inhalation, to the person requiring it, of a gaseous mixture containing from 1 to 80 ppmv of NO and at least 20 vol % of oxygen approximately, preferably at least 21 vol % of oxygen approximately, by means of a gas delivery installation, as described above according to the invention, comprising an NO delivery apparatus for delivering NO at the required dosage, so as to treat (at least partially) said pulmonary hypertension and/or said hypoxia, which can be caused by one or more pulmonary diseases or disorders such as PPHN (persistent pulmonary hypertension of the newborn) or ARDS (acute respiratory distress syndrome) or can be caused by heart surgery with placement of the patient on extracorporeal blood circulation (ECC).

### Definitions

In general, within the context of the invention:
“ppmv” means parts per million by volume.
“vol %” means percentage by volume.
“NO” denotes nitric oxide.
“NO₂” denotes nitrogen dioxide.
“N₂” denotes nitrogen.
“O₂” denotes oxygen.
the pressures are expressed in “bar absolute”, abbreviated to “bar”.
the terms “concentration”, “quantity”, “proportion”, “dose” and “content” are considered as equivalents.
the terms “means of/to/for” are considered to be wholly equivalent to and capable of being substituted by the terms “device of/to/for”, for example the term “control means” may be replaced by “control device”, the term “valve means” may be replaced by “valve device”, the “storage means” may be replaced by “storage device”, etc.
“pressure measurement” is understood as a pressure value (e.g. a numerical value) or a signal representative of such a pressure value, which reflects or corresponds to the gaseous pressure measured by a pressure sensor or the like.
the terms “upstream” and “downstream” are used in relation to the normal direction of gas flow, i.e. in the direction from the gas containers to the delivery apparatus and then to the patient.

### BRIEF DESCRIPTION OF VIEWS OF DRAWINGS

The invention will now be better understood from the following detailed description, provided by way of non-limiting illustration, with reference to the appended figures, in which:

FIG. 1 shows schematically an embodiment of a gas administration installation according to the invention.

FIG. 2 shows schematically an embodiment of the internal architecture of the NO delivery apparatus of a gas administration installation according to the invention, in particular a gas administration installation according to FIG. 1.

### DETAILED DESCRIPTION

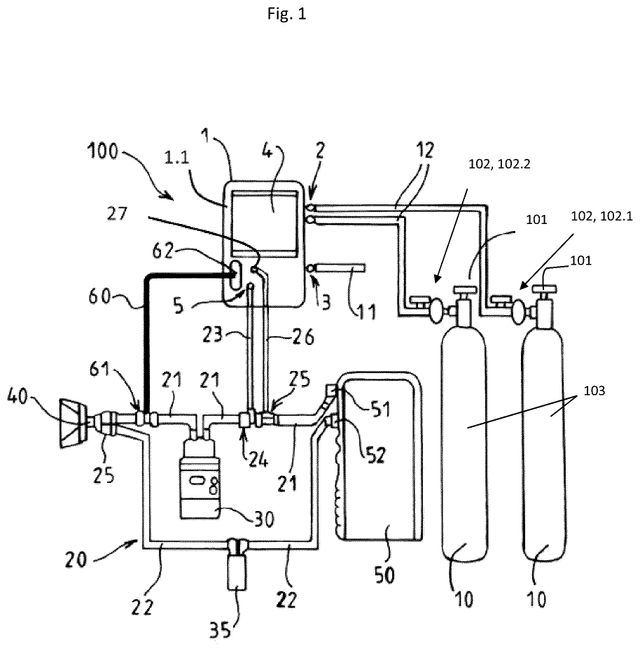

FIG. 1 shows schematically an embodiment of a gas administration installation 100 according to the invention, comprising an NO supply apparatus 1 which supplies a gaseous mixture based on nitric oxide (NO), and a medical ventilator 50 which supplies a gas containing at least 20 vol % of oxygen, such as air or the like.

Here, the installation 100 comprises NO sources 10, namely two pressurized gas containers or cylinders 10.1, 10.2, each containing an NO-based gas, that is to say an NO/N₂ gas mixture, here an NO/N₂ gas mixture containing between 100 and 1000 ppmv of NO (remainder N₂), for example 450 or 800 ppmv of NO (remainder N₂), or any other adequate concentration, which feed an NO/N₂ mixture to the device or apparatus 1 for delivering or supplying NO, making it possible to monitor and control the supply of the NO/N₂ gas mixture.

The NO/N₂ gas mixture is generally conditioned in each container 10.1, 10.2 at a pressure, referred to as “high pressure”, of at least 150 bar, which corresponds to the initial pressure prevailing in each container before any withdrawal begins, that is to say measured before any withdrawal or use of gas (i.e. full container). In some cases, it can be as much as 230 to 250 bar, or even more. Of course, the pressure of the gas in the containers 10.1, 10.2 decreases as the gas is withdrawn, that is to say as the gas is progressively used, thus causing progressive emptying of the containers 10.1, 10.2.

As is illustrated in FIG. 1, each container 10.1, 10.2 comprises a body 103 of generally cylindrical or ogive shape, containing the gas within its internal volume, which body 103 is surmounted by a gas distribution valve 101 serving to control the exit of the gas from the internal volume of the body 103.

In FIG. 1, each valve 101 is what is called a “simple” valve, that is to say one that does not contain internal expansion means. As a result, external expansion means 102, namely first and second expansion means 102.1, 102.2, such as gas pressure reducers, are arranged downstream of each valve 101 in order to ensure a reduction of the gas pressure to a desired expansion pressure (PD) which is adjusted at each pressure reducer, typically an expansion pressure of less than or equal to 10 bar, preferably of less than or equal to 8 bar, typically of between 4 and 7 bar, advantageously of between 5 and 6 bar. The gas pressure reducers can be screwed, for example, onto the gas outlet of the valves 101.

Alternatively, according to another embodiment (not shown), the valves 101 could be of the type with an integrated pressure reducer (RDI), that is to say the expansion means 102 would then be arranged within (i.e. integrated) the body of each valve 101, for example an expansion valve cooperating with a valve seat. In this case, the gas leaving each valve 101 would then be at the desired expansion pressure, i.e. at an expansion pressure of less than or equal to 10 bar, as previously.

The gas cylinders 10.1, 10.2 are each fluidically connected to the NO supply apparatus 1 via gas feed lines 12, typically a first gas feed line 12.1 and a second gas feed line 12.2, such as flexible hoses or conduits or the like, which may be equipped with devices for monitoring the gas pressure, in particular with one or more pressure gauges or the like.

The gas feed lines 12 convey the NO-based gas at the expansion pressure (PD), that is to say the gas expanded within the expansion means 102, i.e. the first 102.1 and second 102.2 pressure reduction means, to the apparatus 1.

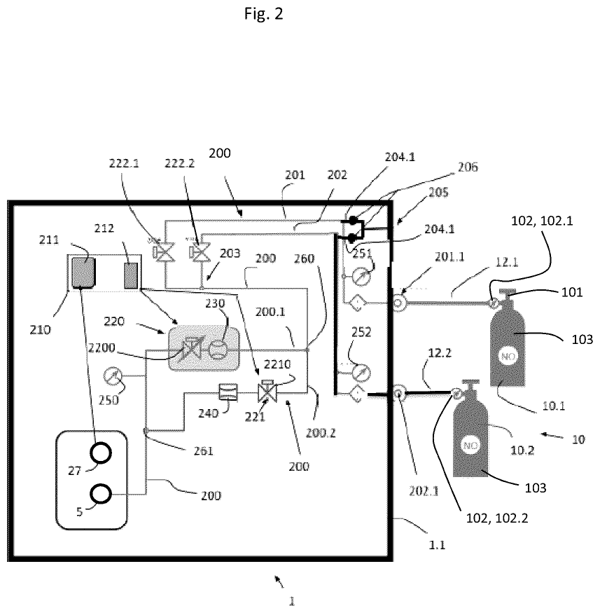

More precisely, the gas feed lines 12 are fluidically connected to gas inlets 2, namely a first and a second gas inlet 201.1, 202.1 of the NO delivery apparatus 1, which supply an internal gas circuit 200, as detailed in FIG. 2, used to convey the gas within the NO supply apparatus 1, i.e. in the external casing or shell 1.1 of the apparatus 1.

In the embodiment of FIG. 2, the internal gas circuit 200 comprises a first circuit section 201, also called the first inlet section, fluidically connected to a first gas inlet 201.1, and a second circuit section 202, also called the second inlet section, fluidically connected to a second gas inlet 202.1 of the apparatus 1. The first and second gas inlets 201.1, 202.1 are also referred to as first and second NO inlets, since they are supplied with NO/N₂ mixture coming from the gas cylinders 10.1, 10.2.

The first circuit section 201 comprises a first valve means 222.1, typically a first control valve, controlled by the control means 210 to control the flow of gas within the first circuit section 201, and the second circuit section 202 comprises a second valve means 222.2, typically a second control valve, controlled by the control means 210 to control the flow of gas within the second circuit section 202; the first and second valve means 222.1, 222.2 are typically solenoid valves or the like.

As is illustrated in FIG. 2, the two circuit sections 201, 202 are arranged in parallel with each other but are fluidically connected to each other at a connection site 203 of the gas circuit 200, which connection site 203 is located downstream of the valve means 222.1, 222.2, considering the direction of flow of the gas in the gas circuit 200, that is to say in the direction from the first and second gas inlets 201.1, 202.1 to the control valves 222.1, 222.2.

The first gas inlet 201.1 is fluidically connected to the first gas feed line 12.1, typically a first flexible hose, while the second gas inlet 201.1 is fluidically connected to the second gas feed line 12.2, typically a second flexible hose. The connections can be provided by conventional connectors.

In general, the gas pressure prevailing in the two circuit sections 201, 202 corresponds to the gas pressure conveyed by the gas feed lines 12.1, 12.2, i.e. to the expansion pressure, as long as the containers feeding these sections contain a sufficient quantity of NO-based gas.

Indeed, each gas feed line 12 contains and conveys NO-based gas at a pressure which is at first equal to the expansion pressure (PD), i.e. less than 10 bar, for example between 4 and 7 bar, and which then becomes lower than the expansion pressure (PD) when the NO container that supplies the gas supply line 12 in question is close to being empty, that is to say when the major part (i.e. almost all) of the gas that it contains has been used.

In other words, the NO-based gas, whether at the expansion pressure (PD) for as long as the container supplying the NO-based gas contains sufficient gas, or at a pressure which is below the expansion pressure and which continues to decrease as the container in question becomes empty, is supplied by one or other of the gas feed lines 12 to one or other of the two circuit sections 201, 202 of the apparatus 1.

The pressure of the gas within the two circuit sections 201, 202 is continuously monitored, i.e. measured, by pressure-measuring means, namely a first and a second pressure-measuring means 251, 252, arranged on the first and second circuit sections 201, 202, respectively, such as pressure sensors arranged in such a way as to be able to carry out pressure measurements within the circuit sections 201, 202 and to supply these measurements to the control means 210, in particular so as to be able to carry out automatic switching from one section to the other 201, 202, when the pressure measured within the considered section 201, 202 becomes less than or equal to the fixed threshold pressure (PS), for example of the order of 4 bar.

This automatic switching is controlled by the control means 210, which act on the valve means 222.1, 222.2.

In fact, the valve means 222.1, 222.2, i.e. control valves or the like, control the passage of the NO/N₂ flow in the two sections 201, 202 to effect an alternating supply to the downstream part of the gas circuit 200 located in particular downstream of the connection site 203, that is to say only one or other of the sections 201, 202 can supply said downstream part of the gas circuit 200 but never both simultaneously, that is to say at the same time.

In other words, when the first valve means 222.1 is controlled by the control means 210 to be in the open position and thus to allow gas to pass, the second valve means 222.2 is (controlled) in the closed position and then prevents any passage of gas, and vice versa. However, it is possible for the two valve means 222.1, 222.2 to both be (controlled) in the closed position in order to block any delivery of the gas into the downstream part of the circuit 200, as is explained below.

Furthermore, it will be seen that the first circuit section 201 and the second circuit section 202 each comprise an exhaust line 204.1, 204.2 to the atmosphere, namely a first 204.1 and a second 204.2 exhaust line to the atmosphere, typically pipes or the like. The exhaust lines 204.1, 204.2 to the atmosphere are fluidically connected to the atmosphere via one or more exhaust ports 205, namely here a single exhaust port 205.

Each exhaust line 204.1, 204.2 to the atmosphere comprises an exhaust valve 206 controlled by the control means 210 so as to control their opening or closing, hence to authorize/allow or prohibit/stop any escape of gas from one or other of the sections 201, 202 to the ambient atmosphere. These exhaust lines 204.1, 204.2 to the atmosphere are used in particular during the phases of purging of the sections 201, 202 and of the flexible hoses connected thereto, i.e. the first and second gas feed lines 12.1, 12.2, as explained below.

As has already been stated, a first pressure-measuring means 251, such as a first pressure sensor, is arranged on the first circuit section 201, in order to measure the pressure of the gas, i.e. NO/N₂, within the first circuit section 201 and, similarly, a second pressure-measuring means 252, such as a second pressure sensor, is arranged on the second circuit section 202, in order to measure the pressure of the gas, i.e. NO/N₂, within the second circuit section 202.

The first and second pressure-measuring means 251, 252, i.e. their pressure taps, are arranged between the NO inlets 201.1, 202.1 and the two valve means 222.1, 222.2 arranged on the circuit sections 201, 202, as can be seen in FIG. 2, preferably near the NO inlets 201.1, 202.1. These pressure-measuring means 251, 252 supply the pressure measurements carried out (i.e. signal or value) to the control means 210, which process them as detailed below.

Conventionally, the control means 210 are electrically connected, via electrical connections or the like, such as electrical cables, to the pressure-measuring means 251, 252, to the exhaust valves 206 and to the valve means 222.1, 222.2, in order to ensure the transfer of data, typically the measurements, and/or control.

The NO delivery apparatus 1 of the installation 100 also comprises an oxygen inlet 3, which is fluidically connected, via an oxygen feed line 11 such as a flexible hose or the like, to an oxygen source (not shown), for example a pressurized oxygen cylinder or a hospital network, that is to say an oxygen supply line arranged in a hospital building. This makes it possible to supply the internal gas circuit 200 with oxygen when necessary.

The medical ventilator 50, i.e. a respiratory assistance apparatus, supplies a flow of oxygen-based respiratory gas, i.e. containing approximately at least 20 vol % of oxygen, preferably approximately at least 21 vol % of oxygen, such as air or an oxygen/nitrogen mixture (N₂/O₂).

The medical ventilator 50 and the NO supply apparatus 1 of the installation 100 are in fluidic communication with a respiratory circuit 20, also called the patient circuit, in particular with a gas feed line or inspiratory branch 21 of the respiratory circuit 20, which serves to convey the gas flow to the respiratory interface 40 supplying the therapeutic gas flow to the patient, that is to say a final gas mixture containing the desired dosage of NO.

More specifically, the final gas mixture to be administered to the patient is formed by mixing the oxygen-based flow (i.e. air or O₂/N₂ mixture) from the medical ventilator 50 and the NO-containing flow, i.e. the NO/N₂ gas mixture, delivered by the NO delivery apparatus 1.

For this purpose, the NO delivery apparatus 1 supplies or injects the NO/N₂ mixture into the respiratory circuit 20, typically into the inspiratory branch 21, via an injection channel or line 23, which fluidically connects the internal gas circuit of the NO supply apparatus 1 to an injection device 24 arranged on the gas supply line 21.

The injection device 24 is configured to mix the NO-containing gas from the NO delivery apparatus 1 with the flow of O₂-containing respiratory gas coming from the ventilator 50 and conveyed by the inspiratory branch 21 of the respiratory circuit 20, and to obtain a combined gas mixture containing NO and oxygen, i.e. the final gas mixture administered to the patient.

More specifically, the injection device 24 comprises a first gas inlet supplied with a flow of O₂-containing respiratory gas from the medical ventilator 50, a second gas inlet supplied with NO-containing gas, i.e. gas coming from the NO delivery apparatus 1, and a gas outlet supplying the combined gas mixture containing NO and oxygen, obtained by mixing, within the injection device 24, of the NO-containing gas with the flow of O₂-containing respiratory gas.

In other words, the flow of NO/N₂ fed by the injection line 23 is then mixed (by virtue of the injection device 24) with the flow of gas based on oxygen (>20% O₂), e.g. air or an oxygen/nitrogen mixture, delivered by the medical ventilator 50 and conveyed by the inspiratory branch 21 of the patient circuit 20, so as to obtain a final mixture, i.e. a combined mixture, which is to be administered to the patient and contains essentially NO at the desired dosage, nitrogen (N₂) and oxygen (O₂), and possibly inevitable impurities (e.g. argon, CO₂, NO₂, etc.), i.e. a final NO/N₂/O₂ gas mixture.

The inspiratory branch 21 of the circuit 20 further comprises a gas humidifier 30 arranged downstream from the injection device 24. It makes it possible to humidify the final gas flow, i.e. the combined NO/N₂/O₂ gas mixture, before it is administered by inhalation to the patient to be treated, by means of a respiratory interface 40, such as a tracheal intubation tube, a breathing mask or the like.

A line for recovering the gases exhaled by the patient forms an expiratory branch 22 of the patient circuit 20. It is fluidically connected to the inspiratory branch 21 via a connection piece 25, such as a Y-shaped piece.

At its upstream end, the inspiratory branch 21 is fluidically connected to an outlet port 51 of the medical ventilator 50, such as a connector, coupling or the like, so as to recover and convey the oxygen-based gas, typically air or N₂/O₂ mixture, supplied by the medical ventilator 50, while the expiratory branch 22 conveying the exhaled gases is fluidically connected to an inlet port 52 of the medical ventilator 50, such as a connector, coupling or the like, so as to return to the medical ventilator 50 all or part of the flow of the gases exhaled by the patient. The expiratory branch 22 can comprise one or more optional components, for example a CO₂ removal device 35, a filter or the like.

Furthermore, a flow rate sensor 25, for example of the hot wire or pressure differential or mass flow type, is arranged on the respiratory circuit 20, in particular on the inspiratory branch 21, between the ventilator 50 and the injection device 24. The flow rate sensor 25 is connected at a connection port to the sensor 27 of the NO delivery apparatus 1 via one or more flow rate measurement lines 26, which are connected at said connection port to the sensor 27. It is used to measure the flow rate of gas delivered by the ventilator 50, such as air or N₂/O₂, circulating in the inspiratory branch 21, upstream from the injection device.

These flow rate measurements carried out by the flow rate sensor 25 make it possible to control or regulate more efficiently the delivery of the flow of NO (i.e. N₂/O₂) by the NO delivery apparatus 1, in particular the flow rate of NO, since the flow rate measurements carried out by the flow rate sensor 25 are returned, via the flow rate measurement line 26 (i.e. electric cables or the like) and the port for connection to the sensor 27, to (micro)processor-based control means 210 of the NO delivery apparatus 1, typically a (micro)controller, which process these flow rate measurements as explained hereinafter and illustrated in FIG. 2. The port for connection to the sensor 27 is connected electrically to the control means 210 via one or more electrical connections, for example electrical cables or the like.

The NO supply apparatus 1 of the installation 100 comprises a rigid shell 1.1, for example made of polymer, comprising the internal gas circuit 200 in FIG. 2, typically gas lines, passages or ducts or the like, serving to convey the flow of NO-based gas, i.e. the NO/N₂ mixture, coming from the cylinders 12 of NO/N₂ mixture. The internal gas circuit 200 fluidically connects the gas inlets 201.1, 202.1 of the NO supply apparatus 1 to the injection line 23, so as to convey the flow of NO-based gas between them.

In the embodiment shown schematically in FIG. 2, a portion of the internal gas circuit 200 comprises two additional gas sections arranged in parallel, i.e. a main section 200.1 and a secondary section 200.2, called the backup section. The main section 200.1 and the secondary section 200.2 are fluidically connected to each other and to the rest of the gas circuit 200 at upstream 260 and downstream 261 connection sites which are situated respectively upstream and downstream from the main and secondary flow rate control means 220, 221.

In this case, in normal operating mode, the NO/N₂ flow passes through the main section 200.1, whereas in the event of a malfunction, for example if the main flow rate control means 220, such as a mass flow controller or MFC, are rendered non-operational or defective, the flow of NO/N₂ can pass through the backup section 200.2.

Of course, according to another embodiment (not shown), the internal gas circuit 200 could be configured differently, for example it could comprise a single gas line instead of the two sections 200.1, 200.2, which line would be used in normal operating mode and in backup mode. However, in this embodiment, malfunctioning of the main flow rate control means 220 could not be taken into account, and the apparatus 1 would then become non-functional.

In general, the main and secondary flow rate control means 220, 221, such as main and secondary valve means 2200, 2210, shown schematically in FIG. 2, i.e. one or more valve devices, for example one or more proportional solenoid valves controlled by the control means 210, are arranged on the internal gas circuit 200, in particular on the main 200.1 and secondary 200.2 sections, and serve to control or adjust the gas flow circulating therein in the direction of the injection line 23, i.e. towards the injection device 24, irrespective of whether this is in normal operating mode or in backup mode.

Preferably, the main section 200.1 comprises a proportional solenoid valve 220 and an additional flow sensor 230, typically a mass flow controller or MFC, whereas the secondary section 200.2 comprises one or more solenoid valves of the all-or-nothing (AON) type 221, preferably controlled in pulsed mode. Preferably, the main and secondary flow control means 220, 221 of the NO supply apparatus 1 are commanded, i.e. controlled, by the control means 210, i.e. one (or more) control device(s) or (micro)controller(s), arranged in the housing 1.1 of the NO supply apparatus 1.

Generally, the control means 210 of the apparatus 1, for example a controller, comprise one or more electronic boards comprising one or more microprocessors 211 implementing one or more algorithms.

The control means 210 make it possible in particular to adjust or control the flow rate of NO-based gas by controlling all or some of the valve means 2200, 2210, typically to open or close one or more (solenoid) valves, for obtaining a flow rate of NO-based gas, typically to permit or stop the flow of gas.

Of course, the control means 210 also make it possible to carry out calculations and/or to control or command all of the electromechanical elements of the apparatus 1, such as solenoid valves, displays, etc.

In particular, during operation, the control means 210 can determine the NO flow rate to be supplied in order to obtain the desired NO content in the combined mixture, i.e. the desired dosage of NO, in particular on the basis of the set NO content regulated and/or fixed by the user, the composition of the gaseous NO/N₂ mixture, in particular the NO content in this gaseous NO/N₂ mixture, and one or more flow rate measurements carried out by the flow sensor 25 arranged on the inspiratory branch 21 and connected by a flow rate measurement line 26 to the NO supply device 1, in particular to the control means 210, via the port for connection to the sensor 27.

The internal gas circuit 200 of the NO supply apparatus 1 can also comprise other elements or components, in particular one or more pressure sensors, one or more additional flow rate sensors or flow meters, and/or calibrated-orifice devices 240 or the like. These other elements can be arranged upstream and/or downstream from the flow rate control means 220, 221, i.e. valve means; for example, it is possible to use an additional flow rate sensor in order to determine the flow rate of NO-based gas circulating in all or part of the internal gas circuit 200, in particular in order to ensure that it conforms with the desired flow rate.

It will be seen in FIG. 2 that the main section 200.1 comprises an additional flow rate sensor 230 arranged upstream from the flow rate control means 220, such as valve means 2200, for example one or more solenoid valves, preferably a proportional solenoid valve, controlling the passage of gas in the main section 200.1. This assembly forms a mass flow controller (MFC).

Furthermore, the secondary section 200.2 comprises a calibrated-orifice device 240 arranged downstream from secondary flow rate control means 221, such as secondary valve means 2210, preferably one or more solenoid valves controlling the flow rate of passage of gas in the secondary section 200.2.

Advantageously, the solenoid valve of the secondary flow rate control means 221 is of the all-or-nothing (AON) type, i.e. able to adopt 2 “stable” positions, namely an open position which allows the gas flow to pass, and a closed position which prevents any circulation of gaseous flow.

Furthermore, the flow meter or additional flow rate sensor 230 of the MFC can be of the pressure differential or mass type, or another type, and cooperates with the control means 210 in order to provide them with flow rate measurements of the NO/N₂ flow.

Usually, the NO supply apparatus 1 also comprises a graphical user interface (GUI) comprising a graphic display screen 4, preferably a touch screen, i.e. a touch panel, serving to display various information items or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

The electrical power for the NO supply apparatus 1, in particular for the components requiring electrical current in order to operate, such as the control means 210, the graphical display screen 4, etc., is provided conventionally by an electrical current source and/or electrical supply means (not shown), for example a connection to the mains current (110/220V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

In addition, the installation 100 also comprises a gas sampling line 60 which fluidically connects the inspiratory branch 21 to the NO supply apparatus 1. It is fluidically connected (at 61) to the gas supply line 21, between the humidifier 30 and the joining piece 25, i.e. the Y-piece, typically in immediate proximity to the joining piece 25, and also to an inlet port 62 of the NO supply device 1, for example a port 62 carried by a connector, coupling or the like, allowing the connection of the gas sampling line 60, such as a flexible hose or the like. The gas sampling line 60 makes it possible to take gas samples and convey them to the NO supply device 1 where they are analysed in an internal gas analyser (not shown), that is to say within a calibration line comprising at least one sensor, in particular one or more electrochemical cells, connected electrically to the control means, in order to verify their conformity. In particular, it should be verified that the composition of the final gas conforms with that of the desired NO/N₂/O₂ gas mixture to be administered to the patient, in particular in order to ensure that it does not contain excessive amounts of toxic NO₂ species, that its oxygen content is not hypoxic, that its NO₂ content is not too high, and that its NO content corresponds to the desired dosage, i.e. the dose of NO to be administered by inhalation, which is usually chosen by the healthcare personnel, i.e. physician or the like. This verification of conformity is conventionally carried out by dedicated measurement means, typically NO₂, NO and O₂ sensors, for example electrochemical cells or the like, which must themselves be calibrated periodically, for example every week. The control means 210 of the apparatus 1 are additionally configured to recover and process, i.e. analyse, the signals coming from the various sensors of the gas analyser, which is arranged in the apparatus 1, and to act in response to these signals, in particular to carry out calibration of the sensors.

The set value of NO and/or the content of NO in the NO/N₂ gas mixture supplying the apparatus 1 can be entered and/or adjusted and/or modified by the user, for example via the HMI, by virtue of dose-regulating means or the like, such as keys, cursors and so on. Preferably, the set value of NO and/or the content of NO in the NO/N₂ gas mixture supplying the apparatus 1 can be stored by the storage means 212 of the apparatus 1.

According to the invention, in order to avoid interruption of the treatment of patients when one of the NO cylinders 10.1, 10.2 is empty, due to the healthcare provider's omission or to their unavailability to switch the supply of the NO supply apparatus 1 from the empty cylinder to the full cylinder, hence in order to improve the safety of treatment of the patients treated by administration of NO gas, the control means 210 are configured to automatically control the first valve means 222.1 and/or the second valve means 222.2 to control the circulation of the gas in the first circuit section 201 and the second circuit section 202 as a function of one or more given pressure threshold values (PS), as has already been explained above.

More specifically, the control means 210 are programmed to allow circulation of gas, i.e. NO/N₂, within the first circuit section 201 and simultaneously to prohibit any circulation of gas within the second circuit section 202 (and vice versa/reciprocally) as long as the gas pressure P measured by the first pressure-measuring means 251, i.e. an instantaneous pressure, is greater than or equal to a given threshold pressure PS, i.e. P≥PS, for example a threshold pressure PS of less than or equal to 4 bar, advantageously between 3 and 4 bar. Of course, another pressure threshold could be chosen.

The threshold pressure value PS can be stored by the storage means 212 of the apparatus 1, such as a computer memory, for example a flash memory, RAM or the like.

In general, when a gas cylinder 10.1, 10.2 is full (before any withdrawal), the gas pressure in the cylinder is generally more than 150 bar, usually at least 180 bar, and can be as high as 300 bar, or more.

When the gas is withdrawn from one or other of the cylinders 10, for example from the cylinder 10.1, it undergoes a pressure reduction within pressure reduction means, such as gas pressure reducers, arranged downstream of the valve fitted to each cylinder or, according to another embodiment, integrated into said valve (i.e. an RDI), up to a desired expansion pressure (PD), typically less than 10 bar, for example between 4 and 7 bar, preferably between 5 and 6 bar.

The reduction in pressure of the NO-containing gas from the first and/or second NO container 10.1, 10.2 to the desired expansion pressure (PD), which is less than 10 bar, is effected by the first and/or second expansion means 102; 102.1, 102.2, such as gas pressure reducers, arranged downstream of the valves 101 fitted to the NO containers 10.1, 10.2.

The gas at the expansion pressure (PD), having passed through the expansion means 102; 102.1, 102.2, is then conveyed to the apparatus 1 via the first gas feed line 12.1 or, as the case may be, via the second gas feed line 12.2, in particular up to the first gas inlet 201.1 of the first circuit section 201 or, as the case may be, to the second gas inlet 202.1 of the second circuit section 202.

As the gas, i.e. NO/N₂, is consumed or used, the first cylinder 10.1 empties, and the pressure of the gas that it supplies tends to decrease progressively until it drops below the expansion pressure value.

The gas at a pressure lower than the expansion pressure (e.g. PD=10 bar) continues to be conveyed, as previously, to the apparatus 1, that is to say to the circuit sections 201, 202.

According to the invention, as has already been mentioned, this progressive decrease in pressure can be monitored by the pressure-measuring means 251, 252, which transmit their measurements to the control means 210.

Consequently, as soon as the gas pressure P measured, for example, by the first pressure-measuring means 251 within the first section 201 becomes lower than the fixed threshold pressure PS, i.e. P<PS, for example less than 4 bar, the control means 210 are configured to interrupt all circulation of gas within the first circuit section 201 and also to allow circulation of gas within the second circuit section 202, this making it possible to guarantee continuity of supply of NO to the internal circuit 200 of the NO delivery apparatus 1.

The gas, i.e. NO/N₂, is then supplied in the same way by the second gas cylinder 10.2, by being expanded at the outlet of the second cylinder 10.2 to the expansion pressure (PD), typically less than 10 bar, which again generates progressive emptying of this second cylinder 10.2, again with a progressive decrease in the gas pressure, in particular in the second section 202, which will drop below the expansion pressure (PD) when the cylinder 10.2 is close to being empty.

This is monitored, as has been explained above, by the control means 210 which receive the pressure measurements P, i.e. instantaneous pressure, from the second pressure-measuring means 252 arranged in the second section 202.

Here again, when the gas pressure P measured by the second pressure-measuring means 252 within the first section 201 becomes lower than the given threshold pressure PS, i.e. P<PS, e.g. below a given threshold pressure PS equal to 4 bar, the control means 210 are configured to interrupt all circulation of gas within the second circuit section 202 and, furthermore, to allow circulation of gas within the first circuit section 201.

In other words, by virtue of the present invention, automatic switching takes place from one section 201, 202 to the other, and this alternately, depending on the gas pressure prevailing therein and on the given threshold pressure PS.

The given threshold pressure value PS, for example 4 bar, can be fixed once and for all and stored or, depending on the case, can be modified, in particular via the HMI of the NO apparatus 1.

Advantageously, the given threshold pressure value PS is between 3 and 6 bar, preferably between 3.5 and 5 bar, more preferably between 3 and 4 bar. Such a pressure value corresponds to a cylinder which is not yet completely empty but will be soon. This residual pressure makes it possible to ensure a switchover of gas supply from the (almost) empty cylinder to a higher-pressure cylinder, typically a (almost) full cylinder, in complete safety for the patient, without interrupting the supply of NO to the patient for the time necessary for this switchover, including during the purge time.

Preferably, after interruption of the circulation of the gas, for example in the first section 201 (or alternatively the second section 202) supplied by the (almost) empty gas cylinder 10.1 but before authorization of the circulation of the gas in the other section, i.e. the second section 202 (or alternatively the first section 201), which is then supplied by the other gas cylinder 10.2, i.e. the full cylinder, the second section 202 is purged, preferably also at least part of the supply line 12.2, such as a flexible conduit, connecting the full gas cylinder 10.2 to the second section 202, in order to rid them of gaseous impurities that may be present there, in particular any toxic compound of the NO₂ type that may be present there as a result of oxidation of NO molecules by oxygen molecules.

Of course, the same is done to purge the first section 201 before conveying gas thereto, when the second gas cylinder 10.2 is almost empty, that is to say when the pressure measured in the second section 202 becomes lower than the threshold pressure PS, e.g. from 3 to 4 bar.

In all cases, the purging is carried out with part of the NO/N₂ mixture from the gas containers 10.1, 10.2. To purge one or other of the sections 201, 202 and the flexible conduit associated with it, i.e. to which it is fluidically connected, the first 204.1 or the second 204.2 exhaust line to the atmosphere is used depending on the section to be purged, namely the first circuit section 201 or the second circuit section 202, respectively, for venting to the atmosphere, via the exhaust orifice 205, the gas that is present therein and at least some of the gaseous impurities that may be present therein, such as toxic NO₂ species.

This purging is carried out by gas flushing with the NO/N₂ mixture from the gas containers 10.1, 10.2, which gas flushing entrains the gaseous impurities, such as toxic NO₂ species, present in the section 201, 202 to be purged and in the flexible conduit associated therewith, and the flow of purging gas thus created is discharged to the outside atmosphere via the exhaust port 205. This purge flow therefore essentially contains nitrogen, NO and possible impurities such as NO₂.

The control means 210 control the one or more exhaust valves 206 arranged on the first 204.1 and/or the second 204.2 exhaust line connected to the section 201, 202 to be purged, so as to control the opening (or closing) thereof, in order to authorize (or prohibit) the escape of the gas coming from the section 201, 202 to be purged, and generally from the gas supply line 12.1, 12.2 connected thereto.

This is able to increase patient safety by eliminating toxic species that may be present in the unused section and its supply line.

Preferably, purging takes place for a given purging duration, typically between 10 and 120 seconds, typically at least 30 seconds. The purging duration is stored, for example by the storage means of the apparatus 1.

A gas administration installation 100 can be used to administer nitric oxide (NO), i.e. the final NO/O₂/N₂ mixture obtained, by inhalation to persons, i.e. patients, suffering from acute pulmonary arterial hypertension, in particular to dilate their pulmonary vessels and increase their oxygenation by improving pulmonary gas exchange, in particular to treat persistent pulmonary arterial hypertension of the newborn (PPHN), acute respiratory distress syndrome (ARDS) observed mainly in adults, or pulmonary hypertension (PH) in cardiac surgery, in adults or children.

What is claimed is:

1. Installation (100) for supplying an NO-containing gas mixture to a patient, comprising:
A) a first and a second NO container (10.1, 10.1) containing an NO-containing gas at a given pressure,
B) an NO delivery apparatus (1) for supplying the NO-containing gas, comprising:
control means (210),
a gas circuit (200) comprising:
a first circuit section (201) comprising a first gas inlet (201.1) configured to fluidically connect thereto the first NO container (10.1) containing the NO-containing gas, and
a second circuit section (202) comprising a second gas inlet (202.1) configured to fluidically connect thereto the second NO container (10.2) containing the NO-containing gas,
a first pressure-measuring means (251), arranged on the first circuit section (201), configured to measure the pressure within the first circuit section (201),
a second pressure-measuring means (252), arranged on the second circuit section (202), configured to measure the pressure within the second circuit section (202),
a first valve means (222.1), arranged on the first circuit section (201), controlled by the control means (210) in order to control the flow of gas within the first circuit section (201), and
a second valve means (222.2), arranged on the second circuit section (202), controlled by the control means (210) in order to control the flow of gas within the second circuit section (202),
C) first and second expansion means (102; 102.1, 102.2) arranged upstream of the NO delivery apparatus (1) and configured to reduce the pressure of the NO-containing gas from the first and second NO containers (10.1, 10.2) to a given expansion pressure (Pd) of less than 10 bar, and
D) a first gas feed line (12.1) and a second gas feed line (12.2), respectively, for conveying the NO-containing gas, which has passed through the expansion means (102; 102.1, 102.2), to the first gas inlet (201.1) of the first circuit section (201) and to the second gas inlet (202.1) of the second circuit section (202), respectively,
characterized in that the control means (210) of the NO delivery apparatus (1) are configured to control the first valve means (222.1) and/or the second valve means (222.2) to:
iii) authorize circulation of gas within one of the first and second circuit sections (201, 202) and simultaneously to prohibit all circulation of gas within the other of said first and second circuit sections (201, 202) as long as the pressure (P) measured by the first or second pressure-measuring means (251, 252) is greater than or equal to a given threshold pressure (PS), i.e. P≥PS, and
iv) when the pressure (P) measured by the first or second pressure-measuring means (251, 252) falls below the given threshold pressure (PS), i.e. P<PS:
a) interrupt all circulation of gas within said first or second circuit section (201, 202) within which gas circulates and
b) authorize circulation of gas within the other of said first or second circuit section (202) within which circulation of gas was prohibited,
where said given threshold pressure (PS) is lower than the expansion pressure (PD).

2. Installation according to claim 1, characterized in that the first circuit section (201) and the second circuit section (202) of the apparatus (1) are connected to each other at a connection site (203) of the gas circuit (200) located downstream of the first and second valve means (222.1, 222.2).

3. Installation according to claim 1, characterized in that the first gas inlet (201.1) of the apparatus (1) is configured to be fluidically connected to the first NO container (10.1) via the first gas feed line (12.1), and the second gas inlet (202.1) of the apparatus (1) is configured to be fluidically connected to the second NO container (10.2) via the second gas feed line (12.2).

4. Installation according to claim 3, characterized in that the first gas feed line (12.1) and the second gas feed line (12.2) of the apparatus (1) comprise flexible hoses.

5. Installation according to claim 1, characterized in that:
a first exhaust line (204.1) to the atmosphere, comprising a first exhaust valve (206.1), is fluidically connected to the first circuit section (201), and
a second exhaust line (204.2) to the atmosphere, comprising a second exhaust valve (206.2), is fluidically connected to the second circuit section (202),
and in which said first and second exhaust valves (206.1) are controlled by the control means (210).

6. Installation according to claim 5, characterized in that the first exhaust line (204.1) to the atmosphere and the second exhaust line (204.2) to the atmosphere communicate with the atmosphere via a single exhaust port (205).

7. Installation according to claim 1, characterized in that the control means (210) are additionally configured to:
control the second exhaust valve (206.2) in order to purge the second circuit section (202), before authorizing gas from the second NO container (10.2) to circulate within said second circuit section (202) or,
alternatively, control the first exhaust valve (206.1) in order to purge the first circuit section (201), before authorizing gas from the first NO container (10.1) to circulate within said first circuit section (201).

8. Installation according to claim 3, characterized in that the control means (210) are additionally configured to purge the first or second circuit section (201, 202) and simultaneously at least a part of the first or second gas feed line (12.1, 12.2) connected to said first or second purged circuit section (201, 202).

9. Installation according to claim 1, characterized in that the expansion pressure (PD) is between 4 and 7 bar, preferably between 4 and 6 bar, more preferably between 5 and 6 bar.

10. Installation according to claim 1, characterized in that the given threshold pressure (PS) is between 2 and 5 bar, preferably between 2 and 4 bar, more preferably between 3 and 4 bar.

11. Installation according to claim 1, characterized in that the given threshold pressure (PS) is stored by storage means (212) of the apparatus (1).

12. Installation according to claim 7, characterized in that:
the control means (210) are additionally configured to perform a purge for a given purge duration, preferably a purge duration of between 10 seconds and 120 seconds, and/or
the purge comprises a gas sweep with the NO-containing gas mixture, typically the NO/N₂ mixture, coming from the first or second NO container (10.1, 10.1).

13. Installation according to claim 1, characterized in that it additionally comprises:
a medical ventilator (50) configured to supply a flow of O₂-containing respiratory gas, and
a respiratory circuit (20; 21) comprising an injection device (24) which is configured to mix the NO-containing gas from the NO delivery apparatus (1) with the flow of O₂-containing respiratory gas supplied by the medical ventilator (50), and to obtain a combined gas mixture containing NO and oxygen.

14. Installation according to claim 1, characterized in that the first and second NO containers (10.1, 10.1) contain an NO/N₂ gas mixture containing between 100 and 2000 ppmv of NO, the remainder being nitrogen (N₂).

15. Installation according to claim 1, characterized in that:
the first and second NO containers (10.1, 10.1) are each equipped with a gas distribution valve (101), and
the expansion means (102; 102.1, 102.2) are integrated in the gas distribution valves (101) or arranged downstream of the gas distribution valves (101) fitted to the first and second NO containers (10.1, 10.1).
